(19) United States
(12) Patent Application Publication
(10) Pub. No.: US 2019/0128587 A1
(43) Pub. Date: May 2, 2019

(54) FILTER FOR USE IN A REFRIGERATOR

(71) Applicant: Christopher Murray, Huntington Beach, CA (US)

(72) Inventor: Christopher Murray, Huntington Beach, CA (US)

(21) Appl. No.: 16/169,724

(22) Filed: Oct. 24, 2018

Related U.S. Application Data
(60) Provisional application No. 62/577,582, filed on Oct. 26, 2017.

(51) Int. Cl. F25D 17/04

(52) U.S. Cl. CPC F25D 17/042; A61L 9/014; F25D 2317/0415

(57) ABSTRACT

A filter for a refrigerator, with the filter including a housing having a main wall and a peripheral wall extending from the main wall to define a cavity. A filter body and a cooling plate may be coupled to the housing and positioned within the cavity. A cover may be coupled to the housing and have at least one inlet opening and at least one outlet opening spaced from the at least one inlet opening. The cover may be transitional between a closed position and an open position. In the closed position, the cover may extend over the cavity and define a fluid flow path from the at least one inlet opening, across the filter body, and through the at least one outlet opening. At least a portion of the cavity may be exposed as the cover transitions from the closed position toward the open position.

### CROSS-REFERENCE TO RELATED APPLICATIONS

This application claims the benefit of Provisional Application No. 62/577,582 filed Oct. 26, 2017, the entire contents of which are incorporated herein by reference.

### STATEMENT RE: FEDERALLY SPONSORED RESEARCH/DEVELOPMENT

Not Applicable

### BACKGROUND

### 1. Technical Field

The present disclosure relates generally to an air filter, and more specifically, to a filter for use within a refrigerator.

### 2. Description of the Related Art

Storing food items within a refrigerator may result in an undesirable odor within the refrigerator.

Accordingly, there is a need in the art for an air filter for use within a refrigerator. Various aspects of the present disclosure address this particular need, as will be discussed in more detail below.

### BRIEF SUMMARY

In accordance with one embodiment of the present disclosure, there is provided a filter sized and structured for use within a refrigerator for filtering the air within the refrigerator. The filter may employ “active” filtration by creating a flow path through which air may flow to achieve filtration, while at the same time cooling the air, which may enhance the overall efficiency of the refrigerator.

According to one embodiment, the filter may include a housing having a main wall and a peripheral wall extending from the main wall to define a cavity, with the housing being placeable within the refrigerator. A filter body may be coupled to the housing and positioned within the cavity. A cooling plate may be coupled to the housing and positioned within the cavity. A cover may be coupled to the housing and have at least one inlet opening and at least one outlet opening spaced from the at least one inlet opening. The cover may be transitional between a closed position and an open position. In the closed position, the cover may extend over the cavity and define a fluid flow path from the at least one inlet opening, across the filter body, and through the at least one outlet opening. At least a portion of the cavity may be exposed as the cover transitions from the closed position toward the open position.

The at least one inlet opening may include a plurality of inlet openings aligned along a common axis. The at least one outlet opening may include a plurality of outlet openings aligned along a common axis.

The cooling plate may be positioned upstream of the filter body.

The cover may be pivotable relative housing between the closed position and the open position.

The housing may be adherable to the refrigerator.

The filter may further comprise a hook coupled to the housing for hanging the housing within the refrigerator.

According to another embodiment, there may be provided a method of deodorizing air within a refrigerator. The method may include receiving air within a plurality of inlet openings formed in a filter. The filter may additionally include a cooling plate, a filter body, and a plurality of outlet openings in spaced relation to the plurality of inlet openings such that a flow passage is formed which extends from the plurality of inlet openings, over the cooling plate, through the filter body and then through the plurality of outlet openings. The method may include cooling of the air by allowing the air to pass over the cooling plate, and deodorizing the air by allowing the air to pass through the filter body.

The present disclosure will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is an upper perspective view of a refrigerator air filter in accordance with an embodiment of the present disclosure, the air filter being in an open configuration;

FIG. 2 is an upper perspective view of the refrigerator air filter in a closed configuration;

FIG. 3 is a cross sectional view of the refrigerator air filter depicting air flow therethrough;

FIG. 4 is an upper perspective view of the refrigerator air filter coupled to an interior wall of a refrigerator; and

FIG. 5 is an upper perspective view of the refrigerator air filter coupled to a rack in the refrigerator.

Common reference numerals are used throughout the drawings and the detailed description to indicate the same elements.

### DETAILED DESCRIPTION

Referring now to the drawings, wherein the showings are for purposes of illustrating a preferred embodiment of the present disclosure only, and are not for purposes of limiting the same, there is depicted an air filter 10 specifically configured and adapted for use in a refrigerator 12 for filtering air within the refrigerator 12. The air filter 10 may be helpful in reducing or eliminating odors within the refrigerator 12. Additionally, the air filter 10 may be helpful in actively forcing air to pass through the filter 10.

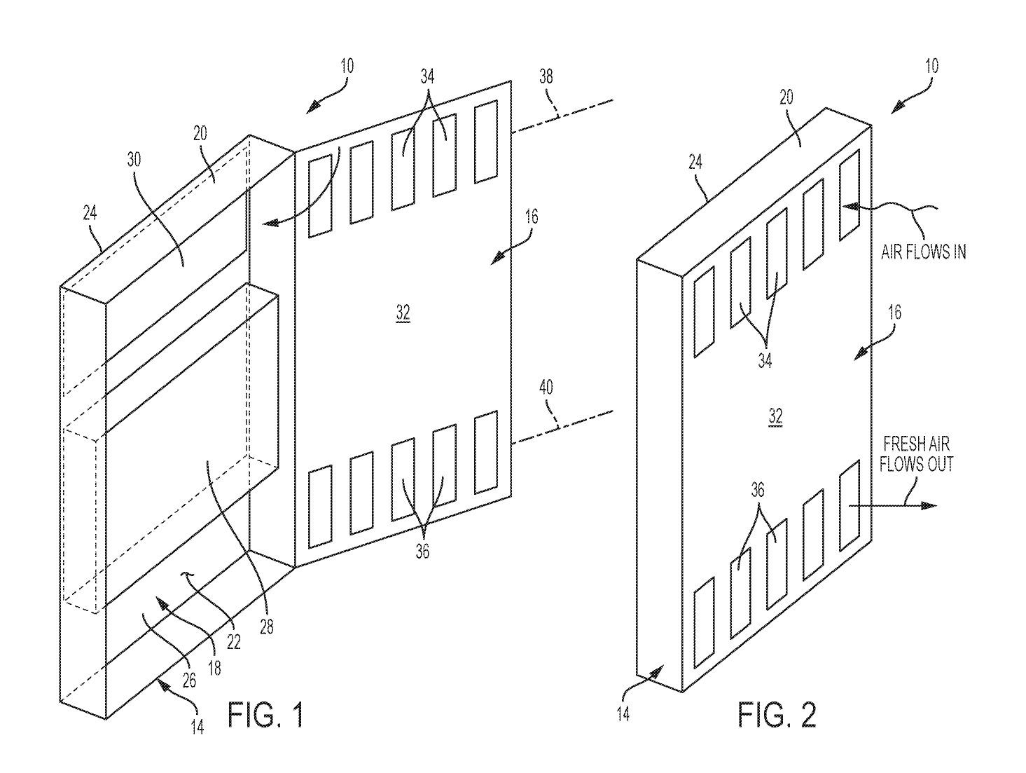

According to one embodiment, the air filter 10 may include a housing 14 and a cover 16 transitional relative to the housing 14 between an open position (see FIG. 1) and a closed position (see FIG. 2), as will be described in more detail below. The housing 14 includes a main wall 18 and a peripheral wall 20 extending from the main wall 18 to define a cavity 22. The main wall 18 may include a planar outer face 24 and an opposing planar inner face 26. The planar configuration of outer face 24 may facilitate mounting of the housing 14 within the refrigerator 12, while the planar configuration of the inner face 26 may facilitate mounting of additional components within the housing 14. The main wall 18 and peripheral wall 20 may be formed via a molding process, and thus, the peripheral wall 20 may be integrally formed with the main wall 18. The main wall 18 and peripheral wall 20 may be formed of a polymer material, metal material, rubber material, or other materials known in the art. In the exemplary embodiment, the main wall 18 and the corresponding peripheral wall 20 are both quadrangular in shape. However, it is contemplated that the scope of the present disclosure is broad enough to encompass additional shapes and configurations of the main wall 18 and peripheral wall 20. For instance, the main wall 18 and peripheral wall 20 may be circular, oval, hexagonal, or any other shape known by those skilled in the art.

Several components may be placed within the housing 14 to achieve desired functionality of the air filter 10. Along these lines, the air filter 10 may include a filter body 28 and a cooling plate 30 positioned within the cavity 22 of housing 14. The filter body 28 may be coupled to the main wall 18 and may filter air which passes through the filter body 28 to deodorize, clean and/or purify the air. In this regard, the filter body 28 may be capable of filtering impurities or other substances in the air which may otherwise create an odor within the refrigerator 12 if not removed from the air. The filter body 28 may be an activated carbon filter body, a charcoal filter body or carbon-based filter body, cotton, wool, or other materials known in the art. The filter body may be porous to allow air to flow therethrough. The exposed surfaces may be impregnated with a material (e.g., activated carbon, baking soda, etc.) which may take away the odor from the air with which it comes in contact. Furthermore, the filter body 28 may be impregnated with a substance to create a desired aroma, such as vanilla, coffee, etc.

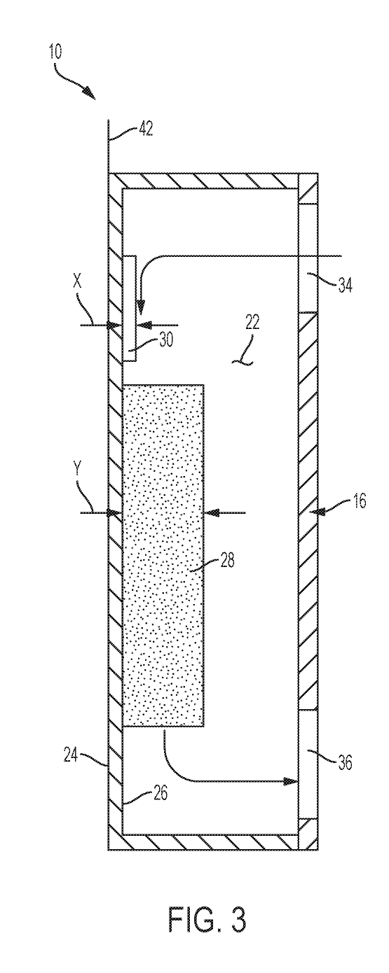

The cooling plate 30 may be coupled to the housing 14 and positioned within the cavity 22 and may cool air that passes over the cooling plate 30. Along these lines, as the air filter 10 resides within the refrigerator 12 over an extended period of time, the cool environment within the refrigerator 12 will cool the cooling plate 30, until the cooling plate 30 reaches substantially the same temperature as the air within the refrigerator 12. Thus, in the event air outside of the refrigerator 12 flows into the refrigerator 12, such as when the door opens, as will be explained in more detail below, the warmer air may flow over the cooling plate 30 to cool the warmer air. The cooling plate 30 may be formed in a variety of different shapes and configurations, and may be located at various locations within the housing 14. For instance, the air filter 10 may include only a single cooling plate 30, or alternatively, several cooling plates 30 may be included within the housing 14. As shown in FIGS. 1 and 3, the cooling plate 30 may be located upstream (e.g., above) of the filter body 28, such that the warmer air which is cooled by the cooling plate 30 may actively flow down through the filter body 28. It is also contemplated that the cooling plate 30 may be downstream (e.g., below) of the filter body 28, or alternatively, in the case of several cooling plates 30, one or more cooling plates 30 may be located upstream of the filter body 28, while one or more cooling plates 30 may be located downstream of the filter body 28. The cooling plate 30 may have thermodynamic properties which allow it to cool down when placed in the refrigerator, and remove heat from warmer air passing over the cooling plate 30. Along these lines, the cooling plate 30 may be formed from a metal material, or other materials known in the art having the desired thermodynamic properties.

Referring specifically to FIG. 3, the filter body 28 and cooling plate 30 may be sized relative to each other to promote air flow across the cooling plate 30 and then through the filter body 28. In this regard, the cooling plate 30 may define a thickness “X” that is smaller than a thickness “Y” of the filter body 28. In other words, an outer face of the cooling plate 30 resides in a plane that intersects the filter body 28, and may reside between opposed inner and outer faces of the filter body 28. The relative thicknesses X, Y of the cooling plate 30 and filter body 28, respectively, allows air to be cooled by the cooling plate 30, and then fall downwardly through the filter body 28, as a result of its increased density attributable to the cooling. The thickness “Y” is shown as being less than a distance between the inner face 26 and the cover body 32. However, it is also contemplated that the thickness “Y” may be equal to the distance between the inner face 26 and the cover body 32.

As noted above, the air filter 10 may include a cover 16 which is coupled to the housing 14. The cover 16 may include a cover body 32 with at least one inlet opening 34 and at least one outlet opening 36 formed in the cover body 32. The cover body 32 may correspond in shape to the housing 14 to allow the cover body 32 to be positioned in opposed relation to the main wall 18 when the cover 16 is in the closed position. Along these lines, in the exemplary embodiment, the cover body 32 is quadrangular in shape and is similar in size to the main wall 18. The inlet openings 34 are formed within the cover body 32 in spaced related to the outlet openings 36. In the exemplary embodiment, the cover 16 includes a plurality of inlet openings 34, i.e., a set of inlet openings, and a plurality of outlet openings 36, i.e., a set of outlet openings. The set of inlet openings 34 are arranged in a first linear array, while the set of outlet openings 36 are arranged in a second linear array. In other words, the first linear array of inlet openings 34 are positioned in spaced relation to each other along a first axis 38, while the second linear array of outlet openings 36 are positioned in spaced relation to each other along a second axis 40. The first axis 38 may be parallel to the second axis 40, and the inlet openings 34 may be positioned adjacent a first end of the cover body 32, while the outlet openings 36 may be positioned adjacent an opposing second end of the cover body 32.

The cover 16 may be transitional relative to the housing 14 between a closed position and an open position. In the closed position (see FIG. 2), the cover body 32 may extend over the cavity 22 in opposed relation to the main wall 18 so as to cover the cavity 22, with the inlet and outlet openings 32, 34 being positioned over the cavity 22. In this regard, when the cover body 32 is in the closed position, a fluid flow path may be defined which extends from the at least one inlet opening 34, into the cavity 22 and across the filter body 28 and cooling plate 30, and through the at least one outlet opening 36. When the cover body 32 is in the closed position, the cover body 32 may be engaged with the peripheral wall 20 such that the primary fluid entry into the cavity 22 is through the inlet openings 34, while the primary fluid exit from the cavity 22 is through the outlet openings 36.

The cover 16 may be transitional from the closed position toward the open position (see FIG. 1) to gain access to the filter body 28 and cooling plate 30. Along these lines, at least a portion of the cavity 22 may be exposed as the cover 16 transitions from the closed position toward the open position. When the cover 16 is in the open position, the cover body 32 is positioned relative to the housing 14 such that the cavity 22 is uncovered and the inlet openings 34 and outlet openings 36 are spaced from the cavity 22, i.e., the inlet openings 34 and outlet openings 36 no longer reside over the cavity 22 and are outside of the footprint defined by the peripheral wall 20.

According to one embodiment, the cover body 32 may be pivotable relative housing 14 about a pivot axis between the closed position and the open position. In this regard, the cover body 32 may be coupled to the housing 14 via a hinge which allows for such pivotal movement.

It is also contemplated that the cover body 32 may be translatable relative to the housing 14 along a translation axis. The translation axis may be generally perpendicular to the pivot axis depicted in FIG. 1, or alternatively, generally parallel to the pivot axis. The air filter 10 may include a slot formed in the peripheral wall, or one or more rails coupled to the peripheral wall to allow for such translatable movement.

The air filter 10 may include one or more features to facilitate mounting of the air filter 10 within the refrigerator 12. For instance, the air filter 10 may include an adhesive layer which is adherable to a wall of the refrigerator 12. As an alternative, the air filter 10 may include a hook 42 or strap, as shown in FIGS. 3 and 5. The hook 42 or strap may allow the air filter 10 to be connected to a shelf located within the refrigerator 12 (see FIG. 5). Other mounting mechanisms known in the art (e.g., suction cups) may also be used for securing the air filter 10 within the refrigerator 12 without departing from the spirit and scope of the present disclosure.

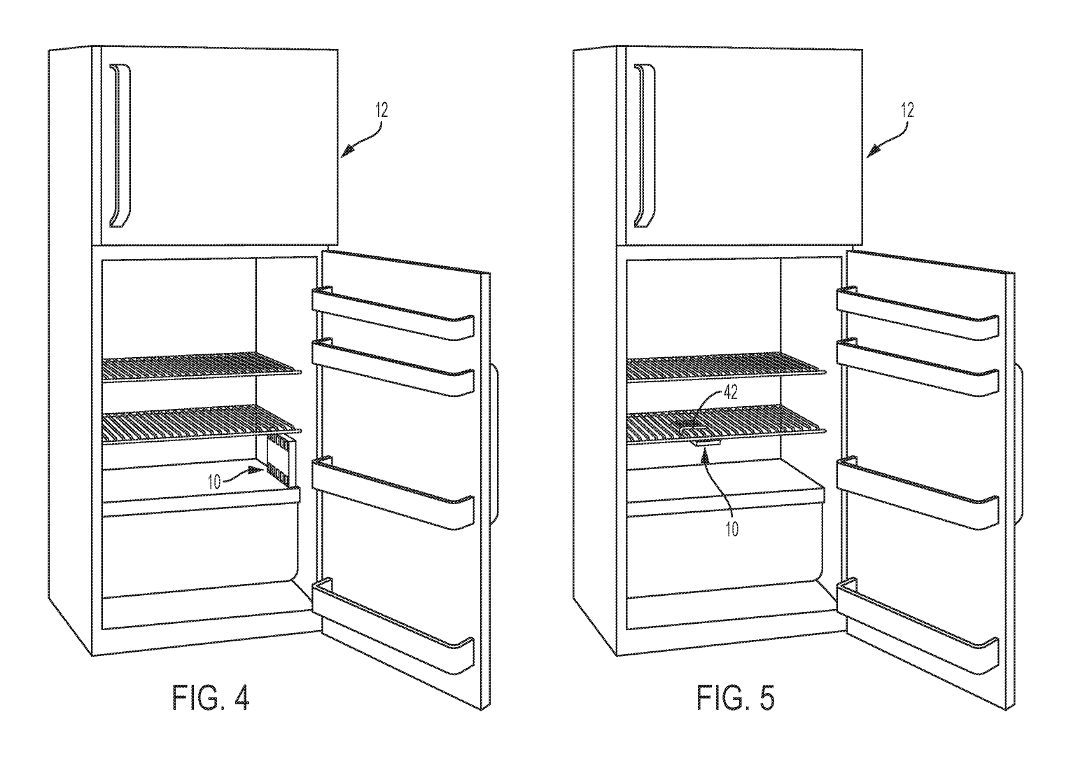

With the basic structure of the air filter 10 described above, the following discussion will focus on an exemplary use of the air filter 10. The air filter 10 may be installed in the refrigerator 12 by adhering the air filter 10 to an internal wall of the refrigerator 12 (see FIG. 4), or by hanging the air filter 10 from a shelf in the refrigerator 12 (see FIG. 5). The air filter 10 may be positioned adjacent the door of the refrigerator 12, where air flow caused by movement of the refrigerator door may be greatest. In this regard, air flow may be greatest adjacent the distal end of the refrigerator door, which is that end of the door opposite the hinge. However, the air filter 10 may be placed anywhere within the refrigerator 12. After the air filter 10 is installed in the refrigerator 12, the temperature of the cooling plate 30 may approach the temperature within the refrigerator 12.

When a user opens the door of the refrigerator 12, warm air may be introduced into the refrigerator 12. The temperature gradient between the cool air temperature within the refrigerator and the warm air temperature outside of the refrigerator may induce air flow through the filter 10, as cold air is less dense than warmer air. In particular, air enters the air filter 10 through the inlet openings 34, and then flows into the cavity 22 to interface with the cooling plate 30 and filter body 28. As the air flows over the cooling plate 30, the temperature gradient between the cooler cooling plate 30 and the warmer air may result in heat transfer from the air to the cooling plate 30, resulting in a decrease in the temperature of the air passing over the cooling plate 30. As shown in FIG. 3, the warm air being cooled by the cooling plate 30 may actively flow downward and through the filter body 28. The air flowing through the filter body 28 may remove impurities and/or deodorize the air. After the air flows over the cooling plate 30 and through the filter body 28, the air may exit the air filter 10 through the plurality of outlet openings 36.

It is also contemplated that air flow through the filter 10 may also be induced by movement of items close to the filter 10. As noted above, swinging of the refrigerator door may induce air movement, as well as moving items within the refrigerator. For instance, a user may shuffle items on the refrigerator shelves, as well as place items within the refrigerator 12 and remove items from the refrigerator. The induced air movement may result in air flow through the filter 10, wherein air flows through the inlet openings 34, across the cooling plate 30, through the filter body 28, and exits through the outlet openings 36.

The air existing the air filter 10 may be cooler, purer, and deodorized relative to the air which enters the air filter 10. In this regard, the air filter 10 may be a dual-purpose filter, which not only filters the air, but also cools the air, resulting in more efficient operation of the refrigerator 12. Furthermore, the inclusion of the inlet and outlet openings 34, 36, coupled with the ability to place the air filter 10 in an air of enhanced air flow allows the air filter 10 to be more “active” than conventional air filters.

The air filter 10 discussed above was discussed as having the housing 14 and the cover 16. However, it is also contemplated that the air filter 10 may the same as discussed above except that the cooling plate 30 and the filter body 28 may be attached to the main wall 18. In this aspect, the housing 14 may only comprise the main wall 18 without the peripheral wall and the cover 16 would not be attached to the housing 14 as well. The air would be freer to pass over the cooling plate 30 because the peripheral wall 20 and the cover 16 does not block the free flow of air. Thus, the air may be more likely to flow downward due to the cooling of the warmer air that might pass over the cooling plate 30 and into the filter body 28.

The particulars shown herein are by way of example only for purposes of illustrative discussion, and are not presented in the cause of providing what is believed to be most useful and readily understood description of the principles and conceptual aspects of the various embodiments of the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A filter for use in a refrigerator, the filter comprising:
a housing having a main wall and a peripheral wall extending from the main wall to define a cavity, the housing being placeable within the refrigerator;
a filter body coupled to the housing and positioned within the cavity;
a cooling plate coupled to the housing and positioned within the cavity; and
a cover coupled to the housing and having at least one inlet opening and at least one outlet opening spaced from the at least one inlet opening;
the cover being transitional between a closed position and an open position, in the closed position, the cover extending over the cavity and defining a fluid flow path from the at least one inlet opening, across the filter body, and through the at least one outlet opening, at least a portion of the cavity being exposed as the cover transitions from the closed position toward the open position.

2. The filter recited in claim 1, wherein the at least one inlet opening includes a plurality of inlet openings aligned along a common axis.

3. The filter recited in claim 1, wherein the at least one outlet opening includes a plurality of outlet openings aligned along a common axis.

4. The filter recited in claim 1, wherein the cooling plate is positioned upstream of the filter body.

5. The filter recited in claim 1, wherein the cover is pivotable relative housing between the closed position and the open position.

6. The filter recited in claim 1, wherein the housing is adherable to the refrigerator.

7. The filter recited in claim 1, further comprising a hook coupled to the housing for hanging the housing within the refrigerator.

8. A method of deodorizing air within a refrigerator, the method comprising the steps of:
receiving air within a plurality of inlet openings formed in a filter, the filter additionally including a cooling plate, a filter body, and a plurality of outlet openings in spaced relation to the plurality of inlet openings such that a flow passage is formed which extends from the plurality of inlet openings, over the cooling plate, through the filter body and then through the plurality of outlet openings;
cooling of the air by allowing the air to pass over the cooling plate; and
deodorizing the air by allowing the air to pass through the filter body.

9. The method recited in claim 8, wherein the cooling step occurs before the deodorizing step.
